(19) United States
(12) Patent Application Publication
(10) Pub. No.: US 2006/0084153 A1
(43) Pub. Date: Apr. 20, 2006

(54) Method of producing diacylglycerides

(76) Inventors: Wuli Bao, Forsyth, IL (US); Mark G. Matlock, Decatur, IL (US); Leif P. Solheim, Decatur, IL (US)

Correspondence Address: KIRKPATRICK & LOCKHART NICHOLSON GRAHAM LLP;HENRY W. OLIVER BUILDING, 535 SMITHFIELD STREET, PITTSBURGH, PA, 15222, US

(21) Appl. No.: 10/966,489

(22) Filed: Oct. 15, 2004

(51) Int. Cl. C12P 7/64

(52) U.S. Cl. 435/134

(57) ABSTRACT

Embodiments of the present invention are directed to a method of producing diacylglycerides comprising mixing one or more fatty acid esters of glycerol and at least one enzyme selected from a lipase and an endo-lipase and transesterifying at least a portion of the one or more fatty acid esters of glycerol to produce diacylglycerides. The enzyme may be a free enzyme or the enzyme may be immobilized. The fatty acid esters of glycerol may comprise at least one of monoacylglycerides, diacylglycerides, and triacylglycerides. Additionally, embodiments of the method of the present invention may comprise mixing at least one of a fatty acid and a fatty acid lower alcohol ester with the one or more fatty acid esters of glycerol and the enzyme. Additional embodiments may comprise mixing glycerol with the one or more fatty acid esters of glycerol and the enzyme.

### TECHNICAL FIELD

This invention relates to a method of preparing glycerides using an enzyme. More particularly, in specific embodiments, this invention relates to a method of preparing diacylglycerides. The method of the present invention comprises preparing glycerides using an enzyme as catalyst, such as, a lipase or an endo-lipase. The enzyme may be immobilized lipase or endo-lipase, or more preferably an immobilized lipase or endo-lipase which preferentially forms diacylglycerides at 1^{st} and 3^{rd} positions of the glyceride.

### BACKGROUND

Presently, edible oils, such as cooking and salad oils, comprise triglycerides as major components. Triacylglycerides (TAG) are fatty acid esters of glycerol formed by esterifying three fatty acids (FA) with glycerol. The fatty acids may have different compositions, such as chain length and degree of saturation, from different source oils. Typical sources of edible oils comprising triacylglycerides are soy oil, rapeseed oil, corn oil, cottonseed oil, safflower oil, sunflower oil, sesame oil, olive oil, and canola oil. However, presently, there is a growing consumer demand for food products with lower concentrations of triacylglycerides for digestive and health reasons. It has been found that is edible oils comprising a high concentration of diglycerides (DAG) meet these consumer demands and are also capable of providing the desired properties of oils for cooking and flavoring of foods.

Diglycerides have been previously prepared by direct esterification of glycerol with fatty acids and by alcohol exchange reactions between glycerol and fats. For example, in the alcohol exchange reaction (glycerolysis) the reaction is carried at a temperature of 200 to 240° C. for 2 to 6 hr. with about 0.1% of a calcium hydroxide catalyst.

The resulting reaction product is a blend of monoglycerides, diglycerides, and triglycerides. The composition of the product is determined by a random distribution of the fatty acids on the three reaction sites of the glycerol. Attempts have been made control the glycerolysis reaction to favor diglyceride production, however, under the specific reaction conditions that minimize triacylglyceride production, production of monoacylglycerides (MAG) are favored and, conversely, conditions that minimize monoacylglyceride production, triacylglyceride production is favored. It is therefore difficult to obtain diacylglycerides in high yield under conventional glycerolysis reactions.

On the other hand, methods for preparation of glycerides using lipase have been proposed. A high quality glyceride may be obtained because the enzymatic catalytic reaction may be conducted at lower temperatures. Lower temperature reaction conditions allow diglyceride formation with less product degradation and, therefore, potentially higher yield. For example, Tsujisaka et al. synthesized glycerides from a fatty acid and glycerol using an aqueous lipase solution of Rhizopus delemar and obtained a composition of 34% monoglyceride, 36% diglyceride and 28% triglyceride with 70% consumption of the fatty acid. See Japanese patent publication 51-7754 (1976).

However, in the disclosed method, the lipase was added as an aqueous solution and adversely affected the yield of ester due to the water reacting with the other reactants in the system. In this case, fatty acids, as well as, the hydroxyl groups of the glycerol remain unreacted; consequently the proportion of glycerol and monoglyceride in the final reaction product was high and, consequently, the yield of diglycerides was low.

Yarmane et al. used lipase of Chromobacterium viscosum var paralipolyticum in a system of 3 to 4% of water to react oleic acid with glycerol to synthesize glyceride. A glyceride composition comprising approximately 36% monoolein, 34% diolein, and 10% triolein was obtained after approximately 80% of the fatty acid was consumed. (JAOCS., 61(4), 776(1984)). However, this method is also impractical because of the high yield of monoglyceride.

Another method of diglyceride production has also been described by Kakuta et al. (Japanese Patent Provisional Publication No. 63(1988)-25987). According to this method, a reaction is carried out essentially without addition of water by using the micro-organism Alcalilipase to increase the yield. According to the description, the glyceride production is the result of the esterification between stearic acid and glycerol using lipase obtained from Alcali genus. The highest reported yield of the reaction resulted in a glyceride composition of 25% monostearin, 70% distearin, 3% tristearin, and 2% stearic acid at an ester yield of 97%. Although the method resulted in a good yield of ester, a considerable amount of monoglyceride was produced and a special commercially unavailable lipase (i.e. Alcalilipase) was required achieve the high yield. This method may, therefore, be impractical for industrial application to obtain high purity diglyceride at a high yield.

Additionally, diglycerides have been prepared by the esterification of fatty acids or a lower alcohol ester of a fatty acid in the presence of an immobilized 1,3 position selective lipase and 1,3 position selective endo-lipase. This reaction requires removing the water or lower alcohol produced from the process to result in high purity diglycerides.

There is a need for a simplified process for the production of diglycerides to allow low cost production of high purity diglycerides in high yield.

### SUMMARY OF THE INVENTION

Embodiments of the present invention are directed to a method of producing diacylglycerides comprising mixing one or more fatty acid esters of glycerol and at least one enzyme selected from a lipase and an endo-lipase and transesterifying at least a portion of the one or more fatty acid esters of glycerol to produce diacylglycerides. The enzyme may be a free enzyme or the enzyme may be immobilized. In certain embodiments of the method, it may be preferable to use an enzyme selected from of a 1,3 position-selective immobilized lipase and 1,3 position-selective immobilized endolipase.

The fatty acid esters of glycerol may comprise at least one of monoacylglycerides, diacylglycerides, and triacylglycerides. Additionally, embodiments of the method of the present invention may comprise mixing at least one of a fatty acid and a fatty acid lower alcohol ester with the one or more fatty acid esters of glycerol and the enzyme. Additional embodiments may comprise mixing glycerol with the one or more fatty acid esters of glycerol and the enzyme.

### DESCRIPTION OF THE INVENTION

The present invention relates to a method for preparing glycerides using an enzyme. More particularly, this invention relates to a method for preparation of diglycerides. The glycerides are produced using an enzyme as catalyst, such as, a lipase or an endo-lipase. The enzyme may be immobilized lipase or endo-lipase, or preferably a lipase or endo-lipase which preferentially forms diacylglycerides at 1^{st} and 3^{rd} positions of the glyceride, herein referred to as a 1,3-position selective immobilized lipase or endo-lipase. The inventors have found that embodiments of the present invention comprising immobilized 1,3 position selective lipase or endo-lipase have excellent transesterification properties for the transesterification (or interesterification) of fatty acid esters of glycerol for the production of diglycerides. Diglycerides can be obtained at a high yield from the method disclosed herein. This reaction has the benefit of not producing a byproduct of water or lower alcohols as compared to the prior art esterification reactions between glycerol and fatty acids and/or lower alcohol esters of fatty acids.

Embodiments of the method of the present invention of preparing a diglyceride may comprise transesterifying at least one fatty acid ester of glycerol in the presence of an lipase or an endo-lipase, preferably a 1,3-position selective immobilized lipase or endo-lipase.

The enzyme for use in the present invention does not have to be immobilized and may be free in the reaction solution. However, the use of such enzymes in organic synthesis, in some cases, may offer operational challenges. For example, free lipase may be denatured by chemical means, such as solvent, product, or substrate interactions, or physical means, such as mechanical agitation. In addition, free enzyme may be more difficult to separate from a reaction solution than an immobilized enzyme. Such problems may be overcome by immobilization of the lipase or endo-lipase. Several immobilized lipases are presently commercially available. The use of immobilized lipases may facilitate the development of continuous, large scale commercial processes and immobilization may also enhance thermal and chemical stability of the lipase.

There are several techniques for producing immobilized lipases any of which may be used in the method of the present invention. For example, the lipase may be immobilized by physical adsorption on various substrates. Lipases may be absorbed on activated carbon, aluminum oxide, celite, cellulose, controlled pore glass, synthetic resins, silica, as well as other substrates. Enzymes may also be immobilized through chemical bonding to substrates. Enzymes may be, for example, ionically or covalently bonded to ion exchange resins, silica, sintered glass, ceramic particles, and polymeric beads, such as polyacrylamide and acrylic resins. Cationic resins, such as carboxymethylcellulose or resins having benzyltrialkylammonium functionality, and anionic resins, such as diethylaminoethyl cellulose may be additionally used for forming immobilized lipases. Enzymes have also been physically entrapped in polymeric materials, such as polyacrylamide beads and sol-gel matrices, also.

Enzymes are a unique class of proteins that may catalyze a broad spectrum of biochemical reactions. Enzymes have traditionally been made in living cells. However, some enzymes may also be produced synthetically. Typically, an enzyme comprises one or more polypeptide chains having a molecular weight that may be greater than ten thousand. Polypeptides are polymers of amino acids, forming chains that may consist of several thousand amino acid residues. The sequence of amino acids in the chain is of critical importance in the biological or chemical functioning of the protein.

An important characteristic of enzymes is their catalytic specificity. A given enzyme may typically catalyze only one particular reaction. For instance, lipases are a specific class of enzymes that catalyze the hydrolysis reaction of fats to glycerol and fatty acids. Lipases may be present in the pancreas, the small intestine and in fatty tissue. Lipases may also be found in milk, wheat germ, and various fungi, for example. Commercially available lipases may be derived from various tissues and organisms, such as, but not limited to, Candida antartica, Candida cylindracea, Candida rugosua, hog pancreas, Aspergilla niger, Mucor miehei, Pseudomonas cepacia, and Pseudomonas fluorencens, and Fusarium solani, for example. Examples of commercially available lipases include, but are not limited to, LYPOZYME™ TL IM, NOVOZYM™ 435, and LYPOZYME™ RM-IM available from NOVOZYME™, as well as others lipases available from NOVZYME™ and lipases from other sources.

Glycerides, or fatty acid esters of glycerol, are compounds of the formula: 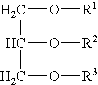
where R¹, R², and R³ are independently selected from H, C₁-C₄ branched or unbranched alkyl group, such as methyl, ethyl, 1-propyl, 2-propyl, butyl, and isobutyl, and aliphatic acyl group of the formula C(═O)R⁴, where R⁴ is a saturated or unsaturated aliphatic C₄-C₂₄, such that at least one of R¹, R², and R³ is C(═O)R⁴. The glyceride is a monoacylglyceride if only one of R¹, R², and R³ is an aliphatic acyl group of the formula C(═O)R⁴; the glyceride is a diacylglyceride if only two of R¹, R², and R³ are aliphatic acyl groups of the formula C(═O)R⁴, and the glyceride is a triacylglyceride if all three of R¹, R², and R³ are aliphatic acyl groups of the formula C(═O)R⁴. R¹ and R³ are in the 1 and 3 positions of the glyceride, therefore, if R¹ and R³ are aliphatic acyl groups and R² is another group, such as H, the glyceride is a 1,3 diacylglyceride.

Fatty acids are carboxylic acids that may be derived from or contained in animal or vegetable fats and oils, for example, soy oil, rapeseed oil, corn oil, cottonseed oil, safflower oil, sunflower oil, sesame oil, olive oil, and canola oil. Fatty acids are typically composed of a saturated or unsaturated chain of alkyl groups containing from 4 to 22 carbon atoms (usually an even number of carbon atoms) and have a terminal carboxyl group, COOH. The carbon atom of the carboxyl group is typically counted in the number of carbons present in the chain of alkyl groups. A fatty acid lower alcohol ester may be formed by the esterification of a fatty acid with a lower alcohol. As used herein, a lower alcohol is an alcohol that comprises lower hydrocarbon chain, such as a C₁-C₄.

In the embodiments of the method of the present invention, there are insufficient acyl aliphatic groups to allow all the glycerol backbones to be converted to triacylglyceridees. In some embodiments, it may be preferred that the molar ratio of acyl aliphatic groups to glycerol backbones (CH₂CHCH₂) is approximately 2 to 1 so the formation of diacylglycerides is preferred. However, the ratio may be higher or lower to control the equilibrium of the reaction as desired, therefore, the molar ratio of acyl aliphatic groups, including all the acyl aliphatic groups on the glycerides and as part of any added fatty acids or fatty acid lower alcohol esters, to glycerol backbones may be between 3 to 1 and 2.5 to 1, or in certain applications between 2.5 to 1 and 2 to 1 or even less than 2 to 1. The molar ratio of aliphatic acyl groups to glycerol backbones may be decreased in the reaction solution by adding additional fatty acid esters of glycerol having an average content of aliphatic acyl groups lower than the average of the reaction solution, by adding monoacylglycerides for example, or simply by adding glycerol to the reaction solution. The ratio may be increased by the opposite procedure, such as adding triacylglycerides, or adding fatty acids or fatty acid lower alcohol esters.

In one embodiment, the method of producing diacylglycerides of the present invention comprises mixing one or more fatty acid esters of glycerol and at least one enzyme selected from a lipase and an endo-lipase. The lipase or endo-lipase catalyzes the transesterification of at least a portion of the one or more fatty acid esters of glycerol to produce diacylglycerides. The reaction may be stopped by separating of the enzyme from the reaction solution or changing the conditions of the reaction to such conditions that the reaction is stopped or the rate of reaction is reduced, such as, for example, by reducing the temperature of the reaction solution or denaturing the enzyme. Separating the enzyme from the reaction solution may be simplified by use of an immobilized lipase.

Further embodiments of the method of the present invention comprise transesterifying the one or more fatty acid esters of glycerol wherein the fatty acid asters of glycerol comprise monoacylglycerides. In this embodiment wherein monoacylglycerides are present, the transesterification reaction between two monoacylglycerides may result in the formation of a diacylglyceride and a glycerol molecule. Of course, the enzyme continues to catalyze the transesterification of the acyl aliphatic groups on the fatty acid esters of glycerol and any formed glycerol molecule until the reaction is stopped. In such embodiments, the fatty acid esters of glycerol may additionally comprise at least one of diacylglycerides, triacylglycerides, fatty acids and fatty acid acyl esters.

Embodiments of the method of the present invention also include transesterifying the one or more fatty acid esters of glycerol comprise a mixture of at least two of monoacylglycerides, diacylglycerides, and triacylglycerides, such as, for example, a mixture of monoacylglycerides and at least one of diacylglycerides and triacylglycerides or a mixture primarily comprising monoacylglycerides and a triacylglycerides. Such mixtures of glycerides may be found in natural sources of oil. The method may further include mixing at least one of a fatty acid and a fatty acid lower alcohol ester with the one or more fatty acid esters of glycerol and the enzyme.

As stated above, a further embodiment of the method of the present invention may comprise mixing glycerol with the one or more fatty acid esters of glycerol and the enzyme. This may be performed to adjust the ratio of glycerol backbones to aliphatic acyl groups in the reaction solution or for any other reason and may be particularly beneficial wherein the fatty acid esters of glycerol comprise triacylglycerides.

Any processing temperature or pressure may be used that results in the transesterification of the fatty acid esters of glycerol. The transesterifying of the one or more fatty acid esters of glycerol may be performed at a pressure from 0.1 to 100 atmospheres, such as at a pressure of less than 20″ Hg vacuum. The present transesterifying may be conducted in bulk or in a solvent such as an alkane. The transesterifying may be conducted at any temperature at which the enzyme is active, such as from 0° to 100°, preferably from 20° to 60°, and most preferably from 40° to 70°.

### EXAMPLES

### Example 1

### Production of DAG from Fatty Acid Methyl Esters (FAME) and MAG

DAG was produced from a mixture of fatty acid methylesters (FAME), 45 grams, distilled monoglycerides (DMG), 55 grams, and NOVOZYM™ 435, 5 grams. NOVOZYM 435 is the lipase B from Candida Antartica commercially available from Novozyme A/S. The FAME and DMG were mixed in a 3 neck flask and heated to 55° Celsius. The enzyme was then added to the flask and the temperature was maintained at about 55° Celsius. The reaction was placed under vacuum (22″ Hg) and sparged with nitrogen gas. Samples were taken every hour after addition of the enzyme. Samples were analyzed by gas chromatograph (“GC”) to quantify the reaction products, byproducts and substrates, see Table 1 for the analytical results.

TABLE 1
DAG production from FAME and MAG

| | Time, hrs | | | | | |
|---|---|---|---|---|---|---|
| | 0 | 1 | 2 | 3 | 4 | 5 |
| FA, % | .90 | 0.80 | 0.27 | 0.42 | 0.21 | 0.18 |
| MAG, % | 48.05 | 17.67 | 17.18 | 14.68 | 11.82 | 10.95 |
| DAG, % | 2.92 | 43.02 | 52.14 | 57.17 | 60.41 | 59.13 |
| TAG, % | Not detected | 0.37 | 4.18 | 8.23 | 14.54 | 19.43 |
| | (N/D) | | | | | |
| FAME, % | 48.13 | 38.14 | 26.23 | 19.50 | 13.03 | 10.31 |
| DAG/TAG | | 115.13 | 12.49 | 6.95 | 4.15 | 3.04 |

As may be seen from Table 1, DAG reached 52% (all percentages are in weight percent unless otherwise indicated) and TAG was 4.18% in the sample after two hours of reaction time. The ratio of DAGFTAG was 12.5. At this same time, the reaction still contained 43% starting material (17% MAG and 26% FAME).

### Example 2

### Production of DAG from FAME and Glycerol

DAG was produced from a mixture of FAME, 90 grams, glycerol, 16.5 grams and NOVOZYM™ 435, 5 grams. The FAME and glycerol were mixed in a 3 neck flask and heated to 55° Celsius. The enzyme was then added to the flask and the temperature was maintained at about 55°. The flask was placed under vacuum (22″ Hg) and sparged with nitrogen gas. Samples were taken every hour after addition of the enzyme. Samples were analyzed by GC to quantify the reaction products, byproducts and substrates, see Table 2.

TABLE 2
Production of DAG from FAME and glycerol

| | Time (hr) | | | | | |
|---|---|---|---|---|---|---|
| | 0 | 1 | 2 | 3 | 4 | 5 |
| FA, % | .12 | 0.40 | 0.33 | 0.35 | 0.36 | 0.44 |
| MAG, % | N/D | 11.78 | 19.91 | 24.55 | 24.74 | 24.70 |
| DAG, % | N/D | 36.55 | 47.92 | 52.09 | 53.07 | 53.87 |
| TAG, % | N/D | 2.02 | 5.56 | 10.98 | 13.57 | 16.40 |
| FAME, % | 99.88 | 49.25 | 26.29 | 12.04 | 8.26 | 4.59 |
| DAG/TAG | | 18.10 | 8.62 | 4.74 | 3.91 | 3.28 |

As may be seen in Table 2, DAG reached 48% in the sample after two hours reaction time. At that time, there was still 26% FAME, the starting material, left in the reaction. In addition, the sample of the reaction medium comprised 20% MAG and 5% TAG.

### Example 3

### Production of DAG from DMG

DAG was produced from a mixture of DMG, 100 grams, and NOVOZYM™ 435, 2 grams. The DMG and glycerol were mixed in a 3 neck flask and heated to 55° Celsius. The enzyme was then added to the flask and the temperature was maintained at about 55°. The flask was placed under vacuum (22″ Hg) and sparged with nitrogen gas. Samples were taken every 30 minutes after addition of the enzyme. Samples were analyzed by GC to quantify the reaction products, byproducts and substrates, see Table 3.

TABLE 3
Production of DAG from DMG

| | Time, min | | | | | | |
|---|---|---|---|---|---|---|---|
| | 0 | 30 | 60 | 90 | 120 | 150 | 180 |
| Glycerol, % | 0.48 | 4.73 | 6.10 | 6.95 | 7.51 | 7.47 | 8.19 |
| FA, % | 0.90 | 1.99 | 1.93 | 1.85 | 1.66 | 1.44 | 1.22 |
| MAG, % | 93.03 | 62.28 | 51.18 | 44.00 | 40.04 | 38.35 | 36.00 |
| DAG, % | 5.55 | 30.87 | 39.88 | 46.03 | 49.15 | 50.69 | 51.33 |
| TAG, % | | | 0.81 | 1.09 | 1.58 | 2.02 | 3.23 |
| Di/Tri | | | 49.14 | 42.14 | 31.07 | 25.12 | 15.91 |
| DAG, mol | 0.09 | 0.51 | 0.66 | 0.77 | 0.82 | 0.84 | 0.86 |
| Glycerol, mol | 0.05 | 0.51 | 0.66 | 0.76 | 0.82 | 0.81 | 0.89 |

As may be seen in Table 2, DAG reached 46% in the sample after 90 minutes of reaction time while there was 44% DMG, the starting material, left in the reaction and TAG was present at 1%. Glycerol was again produced at the same approximately the same molar rate as DAG in the reaction.

### Example 4

### Production of DAG from a Mixture of MAG/DAG/TAG/Glycerol

DAG was produced from a mixture of MAG/DAG/TAG/glycerol (see initial concentrations in Table 4), 100 grams, and NOVOZYM™ 435, 2 grams. The mixture was heated to 55° Celsius. The enzyme was then added to the flask and the temperature was maintained at about 55°. Samples were taken every hour after addition of the enzyme. The samples were analyzed by GC to quantify the reaction products, byproducts and substrates, see Table 4.

TABLE 4
DAG production from a mixture of MAG/DAG/TAG/glycerol

| | Reaction Time, hr | | | | | |
|---|---|---|---|---|---|---|
| | 0 | 1 | 2 | 3 | 4 | 5 |
| Glycerol, % | 8.46 | 5.87 | 4.50 | 4.39 | 3.86 | 4.21 |
| MAG, % | 55.74 | 36.67 | 33.39 | 32.47 | 31.76 | 31.08 |
| DAG, % | 31.65 | 50.37 | 53.66 | 53.49 | 53.69 | 53.28 |
| TAG, % | 3.10 | 4.26 | 5.08 | 5.98 | 7.10 | 7.95 |
| DAG/TAG | 10.22 | 11.81 | 10.57 | 8.95 | 7.57 | 6.70 |

In the sample after 2 hours of reaction time, DAG reached 53%. There was 33% MAG left and the concentration of TAG increase to 5%.

### Example 5

### Production of DAG from Panalite 40 HVK Powder

DAG was produced from a mixture of Panalite powder, 50 grams, and heptane, 100 milliliters, and NOVOZYM™ 435, 1 gram. The Panalite powder and heptane were mixed and heated to 60° Celsius. The enzyme was then added to the flask and the temperature was maintained at about 60° C. Samples were taken every hour after addition of the enzyme. The samples were analyzed by GC to quantify the reaction products, byproducts and substrates. Analytical results showed that after a one-hour reaction, DAG increased and reached 60% from 43%; MAG was decreased to 27% from 47%. TAG was 7.8% and the ratio of DAG to TAG was 7.7.

### Example 6

### Production of DAG from TAG and Glycerol

DAG was produced from a mixture of soy oil, primarily TAG, 20 grams, glycerol, 6 grams and Lypozyme RM-IM, 0.2 grams, a lipase of Rhizomucor miehei commercially available from Novozyme A/S. The reaction medium was placed in a shaker (250 rpm) and maintained at 55° Celsius for 15 hours. A sample of the reaction medium at the end of the fifteen hours was collected and analyzed by GC. The analytical results showed 51% DAG made and 22% TAG left in the reaction. There were also 24.8% MAG and 2.6% fatty acid made in the reaction.

### Example 7

### Production of DAG from TAG and MAG

DAG was produced from a mixture of soy oil, primarily TAG, 15 grams, MAG, 6 grams, and Lypozyme RM-IM, 0.2 grams. The reaction medium was placed in a shaker (250 rpm) and maintained at 55° Celsius for 15 hours. A sample of the reaction medium at the end of the fifteen hours was collected and analyzed by GC. The analytical results showed the final reaction medium comprises 50% DAG, 26% TAG and 20% MAG left in the reaction. There was also 4% fatty acid produced in the reaction.

It is to be understood that the present description illustrates those aspects relevant to a clear understanding of the present invention. Certain aspects that would be apparent to those skilled in the art and that, therefore, would not facilitate a better understanding have not been presented in order to simplify the present disclosure. Although the present disclosure has been described in connection with certain embodiments, those of ordinary skill in the art will, upon considering the foregoing disclosure, recognize that many modifications and variations may be employed. It is intended that the foregoing description and the following claims cover all such variations and modifications.

1. A method of producing diacylglycerides, comprising:
mixing one or more fatty acid esters of glycerol and at least one enzyme selected from a lipase and an endo-lipase; and
transesterifying at least a portion of the one or more fatty acid esters of glycerol to produce diacylglycerides.

2. The method of claim 1, wherein the enzyme is immobilized.

3. The method of claim 2, wherein the enzyme is immobilized on an ion exchange resin.

4. The method of claim 1, wherein the enzyme is selected from of a 1,3 position-selective immobilized lipase and 1,3 position-selective immobilized endolipase.

5. The method of claim 1, wherein the one or more fatty acid esters of glycerol comprise monoacylglycerides.

6. The method of claim 1, wherein the one or more fatty acid esters of glycerol are a mixture of at least two of monoacylglycerides, diacylglycerides, and triacylglycerides.

7. The method of claim 5, further comprising mixing at least one of a fatty acid and a fatty acid lower alcohol ester with the one or more fatty acid esters of glycerol and the enzyme.

8. The method of claim 7, wherein the fatty acid is at least one of a saturated and an unsaturated fatty acid having from 4 to 22 carbon atoms.

9. The method of claim 7, wherein the fatty acid lower alcohol ester is at least one of a lower alcohol ester of one of a saturated and unsaturated fatty acid having from 4 to 22 carbon atoms.

10. The method of claim 7, wherein the one or more fatty acid esters of glycerol further comprise a diacylglyceride.

11. The method of claim 10, wherein the one or more fatty acid esters of glycerol comprise(s) a mixture of a monoacylglyceride and at least one of a diacylglyceride and a triacylglyceride.

12. The method of claim 5, wherein the one or more fatty acid esters of glycerol is/are a mixture of a monoacylglyceride and a triacylglyceride.

13. The method of claim 1, further comprising mixing glycerol with the one or more fatty acid esters of glycerol and the enzyme.

14. The method of claim 13, wherein the one or more fatty acid esters of glycerol comprise(s) a triacylglyceride.

15. The method of claim 14, wherein soy oil is the source of the triacylglyceride.

16. The method of claim 12, wherein the one or more fatty acid esters of glycerol further comprise(s) a diacylglyceride.

17. The method of claim 7, wherein the transesterifying of the one or more fatty acid esters of glycerol is performed under vacuum.

18. The method of claim 17, wherein the transesterifying of the one or more fatty acid esters of glycerol is performed at a pressure of less than 20″ Hg vacuum.

19. The method of claim 1, wherein the transesterifying is performed at a temperature of between 0° C. and 100° C.

20. The method of claim 19, wherein the transesterifying is performed at a temperature of between 40° C. and 70° C.

21. The method of claim 1, wherein the transesterifying is performed in a solvent.

22. The method of claim 21, wherein the solvent is an alkane.
